(19) United States
(12) Patent Application Publication
(10) Pub. No.: US 2006/0184242 A1
(43) Pub. Date: Aug. 17, 2006

(54) Method and apparatus for percutaneous reduction of anterior-posterior diameter of mitral valve

(76) Inventor: Samuel Lichtenstein, Vancouver (CA)

Correspondence Address: SAMUEL LICHTENSTEIN, 4706 DRUMMOND DR., VANCOUVER, BC, V6T-1B4, CA

(21) Appl. No.: 11/400,260

(22) Filed: Apr. 10, 2006

Related U.S. Application Data
(63) Continuation-in-part of application No. 10/690,131, filed on Oct. 20, 2003.

(51) Int. Cl. A61F 2/24

(52) U.S. Cl. 623/2.37; 600/37

(57) ABSTRACT

A method and apparatus for treating mitral regurgitation by approximating the septal and lateral (clinically referred to as anterior and posterior) annulus of the mitral valve. The distal end of the device is inserted into the coronary sinus of the heart and the proximal end of the device rests within the right atrium along the tendon of Todaro and extends to at least the membranous septum of the tricuspid valve. Because the coronary sinus approximates the lateral (posterior) annulus of the mitral valve and the tendon of Todaro approximates the septal (anterior) annulus of the mitral valve, the device encircles approximately one half of the mitral valve annulus. The apparatus is then adapted to deform the underlying structures i.e. the septal annulus and lateral annulus of the mitral valve in order to move the posterior leaflet anteriorly and the anterior leaflet posteriorly and thereby improve leaflet coaptation and eliminate mitral regurgitation.

### FIELD OF THE INVENTION

The present invention generally relates to cardiac surgery, and in particular to mitral valve repair.

### BACKGROUND OF THE INVENTION

Mitral regurgitation with structurally normal leaflets is generally caused by ischemic heart disease and dilated cardiomyopathy. The mitral apparatus is made up of four major structural components and includes the annulus, the two leaflets, the chordae and the papillary muscles. Improper function of any one of these structures or in combination can lead to mitral regurgitation. It is generally believed that acute mitral regurgitation due to myocardial ischemia results from discordant function of the papillary muscles. Annular dilation is a major component in the pathology of mitral regurgitation regardless of causes and is manifested in mitral regurgitation related to dilated cardiomyopathy and chronic mitral regurgitation due to ischemia.

The mitral valve is intended to prevent the regurgitation of blood from the left ventricle into the left atrium when the left ventricle contracts. In a normal mitral valve, the geometry of the mitral valve ensures the cusps overlay each other to preclude the regurgitation of blood during left ventricular contraction and thereby prevent elevation of pulmonary vascular pressures and resultant symptoms of shortness of breath. Studies of the natural history of mitral regurgitation have found that totally asymptomatic patients with severe mitral insufficiency usually progress to severe disability within 5 years. Mitral valve regurgitation requires correction.

At present the treatment consists of either mitral valve repair or replacement, particularly suitable when one of the mitral cusps has been severely damaged or deformed. Both methods require open heart surgery.

Replacement can be performed with either mechanical or biological valves. The mechanical valve carries the risk of thromboembolism and requires anticoagulation with all of its potential hazards, whereas the biological prosthesis suffers from limited durability. Another hazard with replacement is the risk of endocarditis. These risks and other valve related complications are greatly diminished with valve repair.

Mitral valve repair is theoretically possible if the mitral valve leaflets are structurally normal but fail to appropriately coapt because of annular dilatation and/or papillary muscle dysfunction. Various surgical procedures have been developed to improve coaptation of the leaflet and to correct the deformation of the mitral valve annulus and retain the intact natural heart valve function. These procedures generally involve reducing the circumference of the posterior mitral leaflet annulus (lateral annulus) where most of the dilatation occurs regardless of the process since the annulus of the anterior leaflet (septal annulus) does not generally dilate because it is anchored to the fibrous skeleton at the base of the heart. Such techniques generally known as annuloplasty typically suture a prosthesis around the base of the valve leaflets shortening the lateral annulus to reshape the mitral valve annulus and minimize further dilation. Different types of prosthesis have been developed for use in such surgery. In general, prostheses are annular or partially annular shaped and may be formed from rigid or flexible material.

While these methods have been able to successfully treat mitral regurgitation, they have not been without problems and potential adverse consequences. For example, mitral valve annuloplasty fixes the posterior mitral leaflet in a systolic conformation and effectively reduces the mitral valve to a monocusp. In particular the annuloplasty ring prevents the dynamic orifice action of the mitral annulus in diastole and systole.

Miller and associates (J Thorac Cardiovasc Surg 2002;123:881-888; J Heart Valve Disease 2002;11:2-10) studied an open-chest surgical approach of septal-lateral annular cinching with sutures to treat acute ischemic mitral regurgitation. They disclose that a septal-lateral transannular suture was anchored to the midseptal mitral annulus and extermalized to a tourniquet through the midlateral mitral annulus and left ventricular wall. It is experimentally concluded that reduction in mitral annular septal-lateral dimension abolished acute ischemic mitral regurgitation in normal sheep hearts while allowing near-normal mitral annular and posterior leaflet dynamic motion.

In current practice mitral valve surgery requires an extremely invasive approach that includes a chest wall incision, cardiopulmonary bypass, cardiac and pulmonary arrest, and an incision on the heart itself to gain access to the mitral valve. Such a procedure is expensive, requires considerable time, and is associated with high morbidity and mortality. Due to the risks associated with this procedure, many of the sickest patients are denied the potential benefits of surgical correction of mitral regurgitation. In addition, patients with moderate, symptomatic mitral regurgitation are denied early intervention and undergo surgical correction only after the development of cardiac dysfunction. Furthermore, the effectiveness of such procedures is difficult to assess during the procedure and may not be known until a much later time. Hence, the ability to make adjustments to or changes in the prosthesis function to obtain optimum effectiveness is extremely limited. Correction at a later date would require another open heart procedure.

In an attempt to treat mitral regurgitation without the need for cardiopulmonary bypass and without opening the chest, catheter based methods have been devised to repair the valve or place a correcting apparatus for correcting the annulus relaxation. However, none of the prior art discloses a method for effecting a suitable approximation of the septal and lateral annulus of the mitral valve by a device compressing the right atrium against an anchoring point within the coronary sinus, an in particular a device that has a flexible state (for easy introduction) and an adjustable rigid state. The adjustable rigid state allows precise setting of the desired approximation while monitoring mitral valve performance.

Prior art devices can be generally grouped into two types:

devices deforming (mainly shortening) the coronary sinus

devices pulling together two anchor points in order to affect the mitral valve, one of the anchor points can be the coronary sinus (typically using a wire that is pulled and secured).

The devices of the first type, while suitable for percutaneous procedures, are not effective in controlling the leakage of the mitral valve as the forces are not applied from the correct opposite sides of the valve, which are the lateral annulus and the septal annulus. The prior art devices of the second type are not easily adapted to a percutaneous procedure. In order to achieve shortening in the direction connecting the lateral annulus to the septal annulus the anchor points have to be located along this line, so pulling them together will affect the desired direction of shortening. Pulling applied along a different direction will distort the mitral valve but will not achieve the optimal approximation of the two leaflets. The preferred embodiment of the present invention relies on compression rather than tension, making it more suitable for percutaneous application.

The present invention overcomes these shortcomings enabling a percutaneous procedure which is fully adjustable and affecting the shortening in the optimal direction. An additional advantage of the present invention is that the device is removable, as it does not rely on permanent anchor points. Still a further advantage of the present invention is that the device is also adjustable (and removable) at a later date, should further degradation happen in the mitral valve.

### SUMMARY OF THE INVENTION

In general, it is an object of the present invention to provide a method and a device which is deployed in the coronary sinus and right atrium for effecting a 5-10 mm approximation of the septal annulus and lateral annulus of the mitral valve and promote coaptation of the mitral leaflets and dynamic function of the mitral valve annulus. Key to the method of the invention is appreciation that the anterior leaflet of mitral valve is not in same plane as tricuspid valve but sits close to the base of a heart and can be compressed from the right atrial side by applying pressure on the atrial septum in certain particular locations.

Some aspects of the invention relate to a device system for treating mitral regurgitation comprising an elongate element having a first end member and an opposite second end member, wherein the first end member is deployed in a coronary sinus and the second end member is deployed in a right atrium sized and configured for effecting an approximation of a septal annulus and a lateral annulus of the mitral valve. In one embodiment, the approximation is between about 1 and 20 mm, preferably between about 5 and 10 mm.

In one embodiment, the first end member of the elongate element is configured bendable that enables anchoring the first end member in the coronary sinus. In another embodiment, the first member is connected to the second end member of the elongate element by an adjustment system that is configured to allow approximation of the first and second members.

In the preferred embodiment, the elongate element is made of rigid sections and it is continuously adjustable by tightening and loosening a cable joining the section. Adjustment can be done while monitoring valve leakage using Doppler ultrasound, listening to the heart murmur or similar technique.

In operations, the invention is introduced percutaneously via a catheter using an introducer, also serving as an adjustment tool. The elongate element is releasibly coupled to the introducer. After adjustment the introducer is withdrawn.

Some aspects of the invention relate to a method for effecting an approximation of a septal annulus and a lateral annulus of a mitral valve comprising: (a) providing a device having an elongate element and an introducer within a catheter sheath, wherein the elongate element comprises a first end member and an opposite second end member; (b) delivering the catheter sheath endoluminally to a location adjacent the mitral valve; (c) deploying the first end member of the element out of the sheath and placing the first end member in a coronary sinus; and (d) deploying the second end member of the element out of the sheath and placing the second end member in a right atrium. In one embodiment, the step of deploying the second end member is carried out by placing the second end member at extent of the tendon of Todaro in the right atrium.

### BRIEF DESCRIPTION OF THE DRAWINGS

Additional objects and features of the present invention will become more apparent and the invention itself will be best understood from the following Detailed Description of the Exemplary Embodiments, when read with reference to the accompanying drawings.

FIG. 1 shows a cutaway schematic of the heart showing the chambers and the spatial relationships of the various anatomical features discussed in the invention.

FIG. 2 shows a diagram of the triangle of Koch within the right atrium.

FIG. 3 shows a diagram of the heart showing relation of coronary sinus and anterior mitral annulus on a lateral annulus side.

FIG. 4 shows anatomic aspects of the right atrium, as seen at operation.

FIG. 5 shows a diagram of the right heart and planes of tricuspid valve and mitral valve.

FIG. 6 shows one embodiment of a device with compression members applying pressure to lateral annulus and septal annulus according to the principles of the present invention.

FIG. 7 shows a diagram of the compression device placed around the lateral annulus and septal annulus of the mitral valve.

FIG. 8 shows a diagram of a cutaway heart showing a first compression member of the device in coronary sinus exerting force toward the septal annulus while a second compression member of the device in right atrium on tendon of Todaro exerting force toward lateral annulus.

FIG. 9 shows one embodiment of the medical device having a ratchet system.

FIG. 10 shows one embodiment of the medical device having a septal-lateral annular cinching system.

FIG. 11 shows one embodiment of the procedure by using a device comprising a flexible chain of elements capable of being made rigid and adjusted by tightening of a cable.

FIG. 12 shows one embodiment of the device of FIG. 11.

FIG. 13 shows an enlarged view of the device of FIG. 11.

FIG. 14 shows a diagram of the septal-lateral annular cinching device placed across the lateral annulus and septal annulus of the mitral valve.

FIG. 15 shows a four-chamber tomographic view through the aortic root showing the location of the second compression member of the compression device in relation to the interventricular and atrioventricular septum.

### DETAILED DESCRIPTION OF THE EXEMPLARY EMBODIMENTS

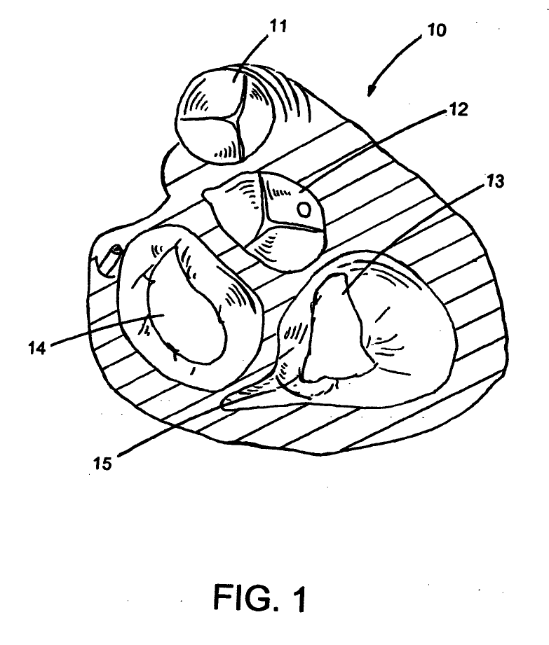

FIGS. 1-15 show a device system and methods for treating mitral regurgitation by approximating the septal and lateral (clinically referred to as anterior and posterior) annuli of the mitral valve. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

The present invention provides an improved apparatus and method to treat mitral regurgitation. Of particular importance and a salient aspect of the present invention allows mitral regurgitation to be treated without resorting to open heart surgery. This is rendered possible not only by the realization that the coronary sinus of a heart is near to and at least partially encircles the lateral mitral valve annulus but more importantly the mitral valve lies in a plane lateral to the right atrial tricuspid valve and as such the triangle of Koch and in particular the tendon of Todaro up to the point of the membranous septum overlies the septal annulus of the mitral valve. Therefore, the device of the present invention may be employed by introduction into the coronary sinus and approximating the extent of the tendon of Todaro in the right atrium to advantageously affect the geometry of the mitral valve annulus by bringing the lateral annulus and septal annulus of the mitral valve into closer proximity and to ensure coaptation of the leaflets.

FIG. 1 shows a cutaway schematic of the heart showing the chambers and the spatial relationships of the various anatomical features discussed in the invention. The heart 10 comprises a pulmonary valve 11, an aortic valve 12, an atrioventricular (also known as tricuspid) valve 13, and a mitral valve 14 when the cardiac valves are in a filling phase (diastole). An opening 15 of coronary sinus (also known as ostium) is also shown in FIG. 1.

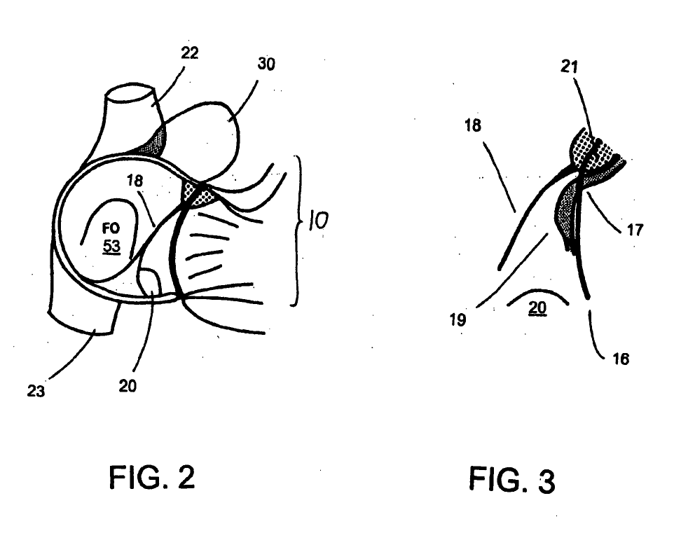

FIG. 2 shows a diagram of the triangle of Koch within the right atrium while FIG. 3 shows a diagram of the heart showing relation of coronary sinus and anterior mitral annulus on a lateral annulus side. The triangle is defined by the tendon of Todaro 18, the orifice of the coronary sinus 20 and the tricuspid annulus 16. Blood from peripheral circulation returns to the right atrium 30 of the heart 10 via superior vena cava 22 or inferior vena cava 23. The diagram shows the relationship of the AV node 19 and AV bundle 17 to triangle of Koch. The membranous septum 21 lies at about the end of Todaro 18.

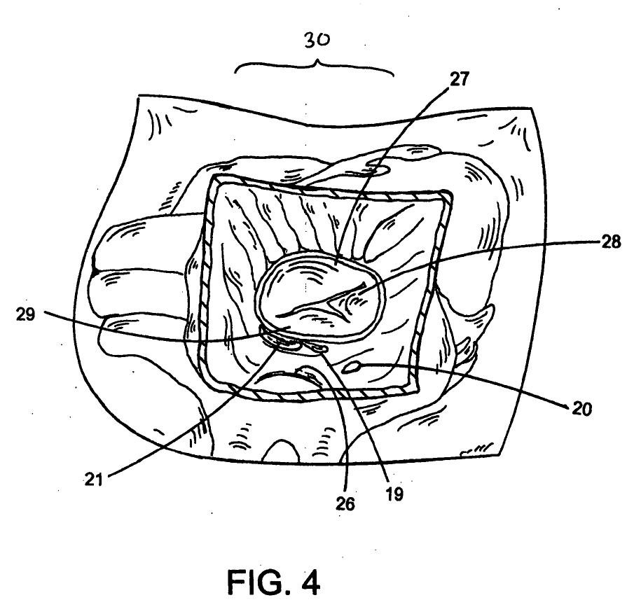

FIG. 4 shows anatomic aspects of the interior of the right atrium 30, as seen at operation. The membranous septum 21 is easily visualized. The tricuspid valve comprises an anterior leaflet 27, a posterior leaflet 28, and a septal leaflet 29. Indentation 26 of anterior (septal) mitral annulus is shown at close to the membranous septum 21.

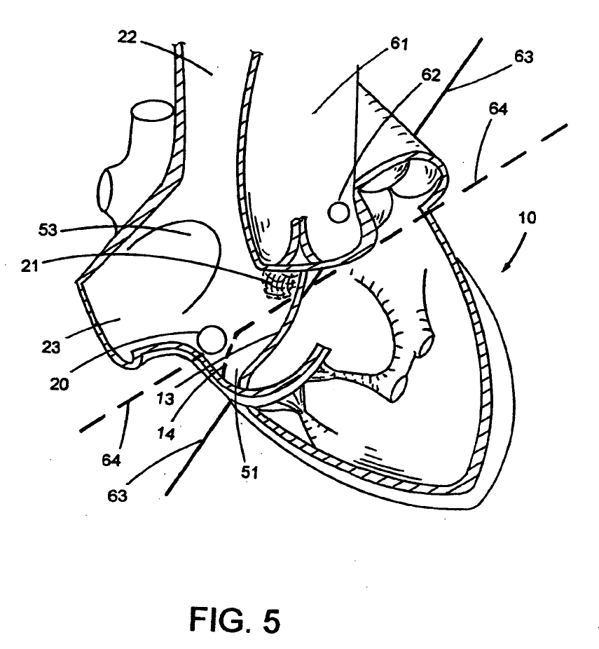

FIG. 5 shows a diagram of the right heart, aorta 61, right coronary 62, fossa ovalis 53, and planes of the tricuspid valve and the mitral valve. The plane 64 of the mitral valve attachment (dashed line) corresponds to the atrial edge of the muscular atrioventricular septum 51 and the inferior edge of the membranous septum 21. The plane 64 of the mitral valve (dashed line) differs from the plane 63 of the tricuspid valve (solid line).

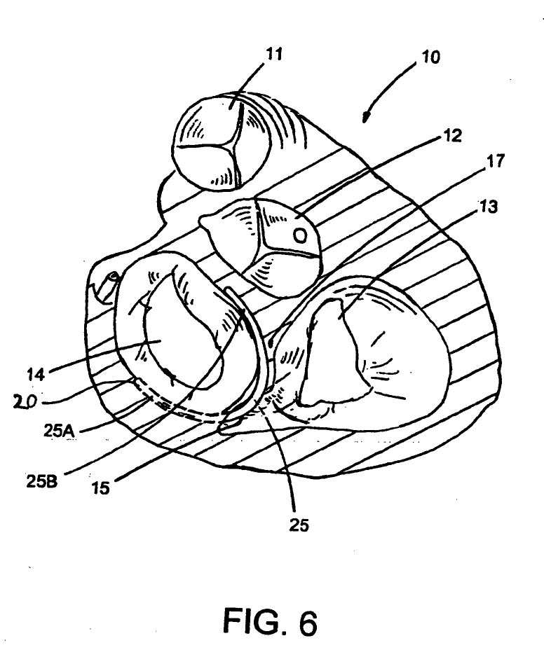

FIG. 6 shows one embodiment of a device with compression members applying pressure to lateral annulus and septal annulus according to the principles of the present invention. Some aspects of the invention provide a device system for treating mitral regurgitation comprising an elongate element 25 having a first end member 25A and an opposite second end member 25B, wherein the first end member 25A is deployed in a coronary sinus 20 through an opening 15 of the coronary sinus, and the second end member 25B is deployed in a right atrium 30 sized and configured for effecting an approximation of a septal annulus and a lateral annulus of the mitral valve 14. The second end member 25B is preferably placed at about the tendon of Todaro. Member 25 can be made from any bendable material that will retain is shape, such as metal or polymer coated metal wire. A good choice of metal is soft (i.e. annealed) type 316 stainless steel wire, about 2 mm in diameter. It is well known is the art that such devices can be coated to give them anti-clotting properties or drug eluting properties, as is the standard practice with coronary stents.

In a different embodiment member 25 is elastic and pre-formed to the correct shape. It is bent for ease of introduction, but once released, it attempts to assumes its natural position. In such a case the preferred material is any flexible material not prone to fatigue such as Nitinol, spring tempered stainless steel, plated beryllium copper or a polymeric material.

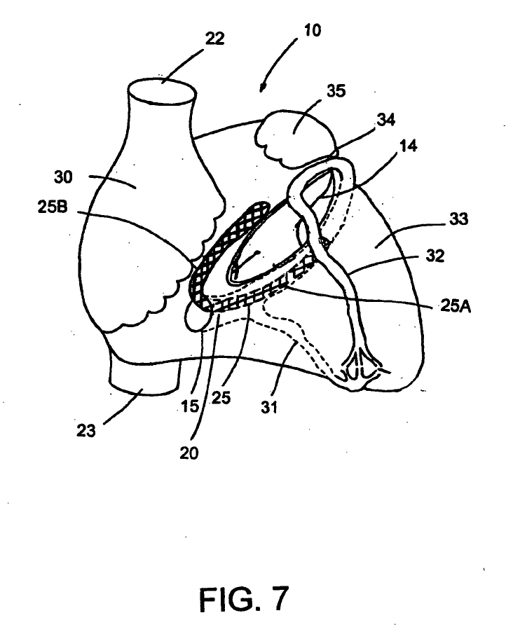

FIG. 7 shows a schematic view of the heart 10, having a compression device 25 positioned therein. The heart 10 generally comprises a right atrium 30, in communication with the superior vena cava 22 and inferior vena cava 23. The left ventricle 33 is positioned below the left atrial appendage 35. Relevant portions of the coronary vasculature include the coronary sinus 20, which extends from the ostium 15 to the junction 34 of the coronary sinus and the great cardiac vein 32.

Because the coronary sinus approximates the lateral (posterior) annulus of the mitral valve and the tendon of Todaro approximates the septal (anterior) annulus of the mitral valve, the device encircles approximately one half of the mitral valve annulus. The apparatus is then adapted to deform the underlying structures i.e. the septal annulus and lateral annulus of the mitral valve in order to move the posterior leaflet anteriorally and the anterior leaflet posteriorly and thereby improve leaflet coaptation and eliminate mitral regurgitation.

One possible method in installing the device from the outside of the heart is to make a cut in the coronary sinus (which is visible from the outside of the heart), insert compression device 25 and close the opening using well known methods such as sutures. The device can be adjusted from the outside of the heart by compressing the heart sufficiently to bend member 25. This is best done while monitoring mitral valve leakage using Doppler ultrasound or any other method.

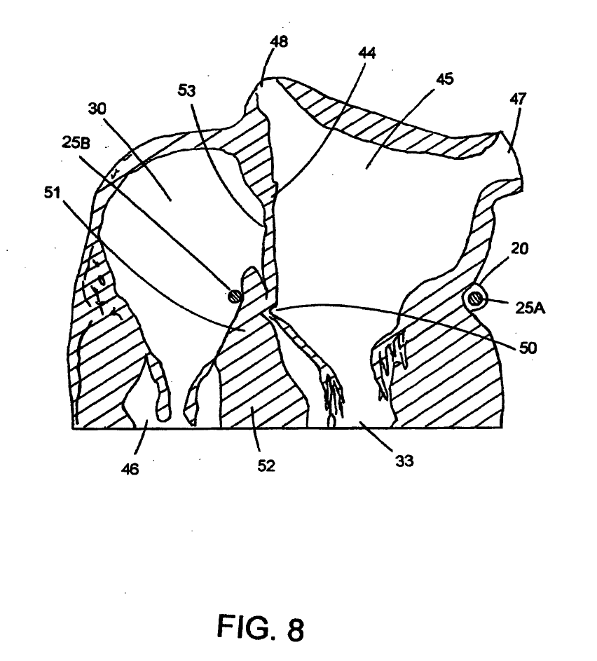

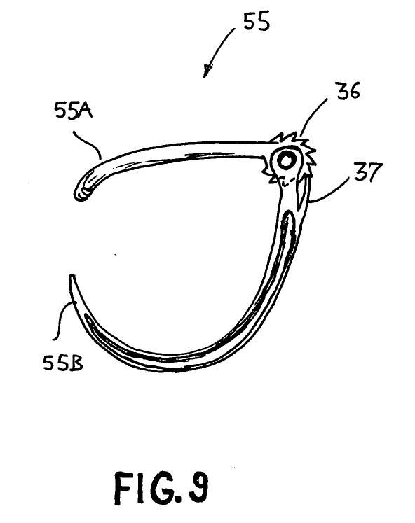

FIG. 8 shows a diagram of a cutaway heart showing a four-chamber view and a first compression end member 25A of the device in coronary sinus 20 exerting force toward the lateral annulus while a second compression end member 25B of the device in the right atrium on tendon of Todaro (or adjacent to tendon of Todaro) exerting force toward anterior annulus. The tomographic view of FIG. 8 shows the relative locations of an interatrial septum 44 (between a right atrium 30 and a left atrium 45), an atrioventricular septum 51, an interventricular septum 52 (between a right ventricle 46 and a left ventricle 33), a left lower pulmonary vein 47 and a right lower pulmonary vein 48. FIG. 8 also shows the anatomic location of septal insertion 50 of the mitral valve and fossa ovalis 53.

FIG. 9 shows a different form of such a device where a ratchet is use for precise adjustment instead of bending or elastic action. Device 55 consists of two parts, 55A and 55B. They are joined by a hinge 36 having teeth at the periphery. A pawl 37 engages said teeth 36. The teeth on hinge 36 can be of saw-tooth shape, only allowing one way motion, or symmetrical shape, allowing stepped (i.e. one tooth at a time) motion in both directions. Such detent action is convenient for precise and repeatable adjustment, as the tactile feel of the detents allows the surgeon to know the shape of the device. In the preferred embodiment pawl 37 forms an integral part of part 55B. The device can be made of injection molded polymer, assembled by snapping together parts 55A and 55B. It can also be made of metal such as type 316 stainless steel. The cross section of part 55A can be round, however it is desired to make the cross section of part 55B in the form of the letter H in order to provide a good passage for the blood stream in the coronary sinus. Parts 55A and 55B can be installed separately, then snapped together in place. This is an advantage when inserting the device via a cut in the coronary sinus. The compression device 55 is a longitudinal dimension having a semi-circular or curved configuration when deployed for encircling at least half of the mitral valve annulus and exerting an inward pressure on not only the lateral (posterior) annulus but also on the septal (anterior) annulus. The inward pressure brings the lateral annulus into closer proximity with the septal annulus. This serves to essentially restore the mitral valve geometry and to promote effective valve sealing action through coaptation of the leaflets to eliminate mitral regurgitation and preserve the dynamic function of the mitral annulus during systole and diastole.

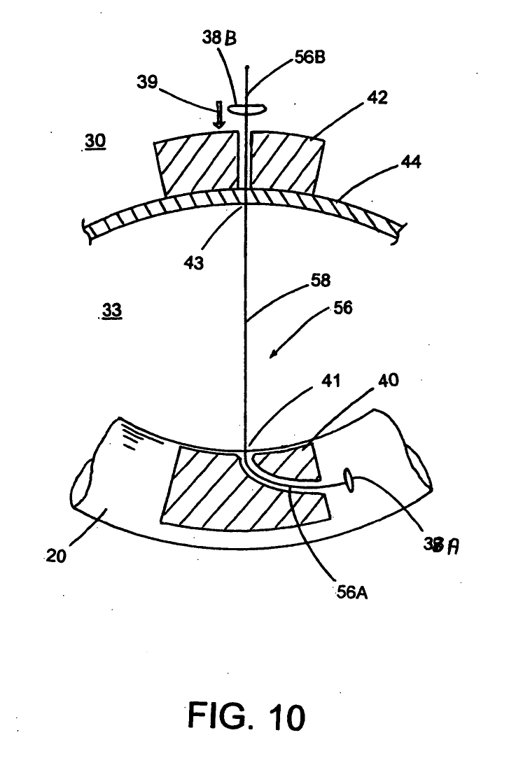

FIG. 10 shows an alternate embodiment of the medical device having a septal-lateral annular cinching system enabling effecting a suitable approximation of the septal annulus and lateral annulus of the mitral valve. The device 56 comprises a first end member 56A and a second end member 56B, wherein the first end member has a first end stopper 38A and the second end member has an axially adjustable second end stopper 38B. By moving the second end stopper 38B toward (as shown by an arrow 39) the first end stopper 38A along the cinching wire 56, the interatrial septum 44 is moved toward the coronary sinus 20 that translates to approximation of the septal annulus and lateral annulus of the mitral valve. In another embodiment, a first short pledget-like member 40 may be introduced into the coronary sinus which will direct the penetrating wire 58 to perforate the left atrial wall 41 of the coronary sinus 20 and enter the left atrium. This wire can then be directed to perforate at a point 43 on the interatrial septum 44 just lateral to the tendon of Todaro and engage in a receiving pledget-like member 42 on the right atrial side of the intra-atrial septum. Once engaged the wire can be cinched so that the septal and lateral annulus of the mitral valve are brought into closer proximity and the reduction in mitral regurgitation observed.

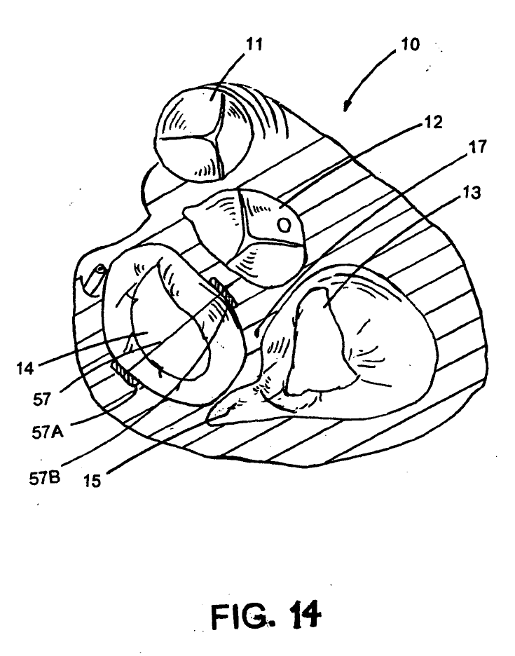

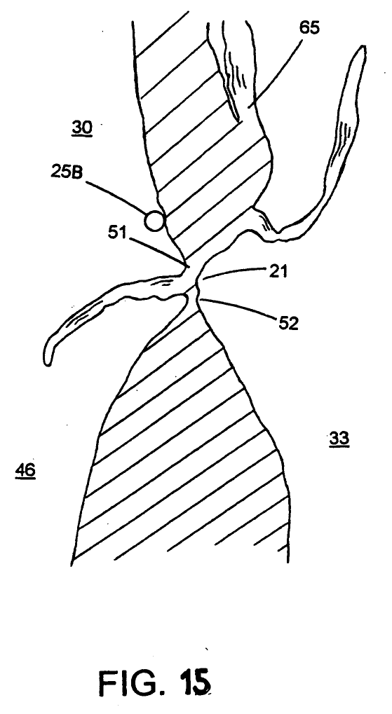

FIG. 14 shows a diagram of the septal-lateral annular cinching device placed across the lateral annulus and septal annulus of the mitral valve. In one particular embodiment as shown in FIG. 14, a cinching device 57 for effecting the condition of septal to lateral annular cinching includes a first end member 57A having a cross-sectional dimension for being deployed within the coronary sinus of the heart and a second end member 57B approximating the extent of the tendon of Todaro within the right atrium. A cinching means for shortening the distance between the end members 57A and 57B is attachably connected to both end members. By appropriate cinching, a suitable approximation of the septal and lateral annuli of the mitral valve is effected. This may be done surgically from lateral wall of heart to inside of right atrium.

Member 57 can be elastic, made of nitinol or other suitable material and takes on a preformed configuration when deployed but is resilient and permits straightening during implantation. Once implanted in the coronary sinus and right atrium the member exerts an inward compressive force on the septal and lateral annulus. However, the preferred embodiment relies on adjustable devices, particularly those than have two states: a flexible state and a more rigid adjustable state. The greatest benefit is achieved when these devices are adjusted while monitoring valve operation

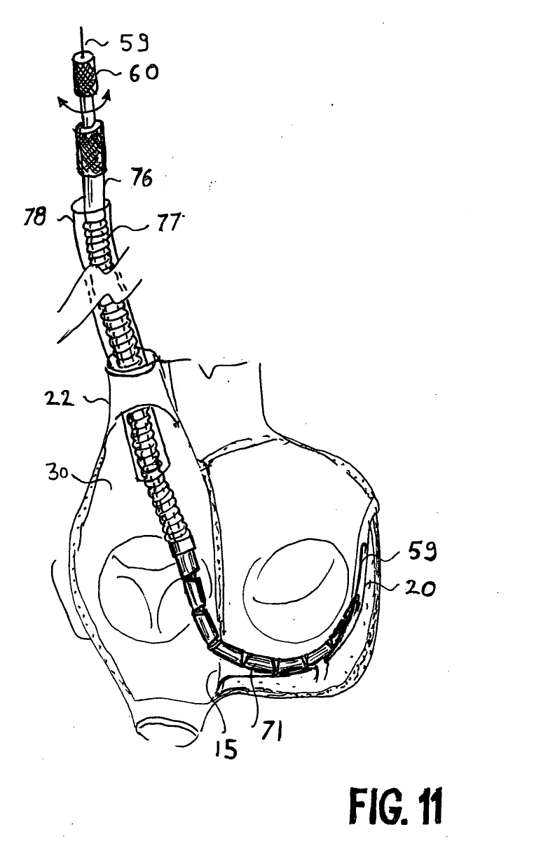

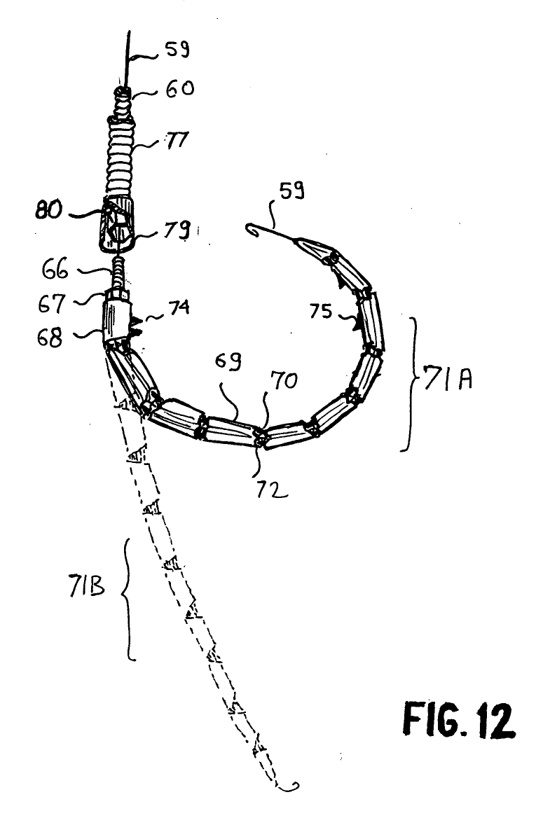

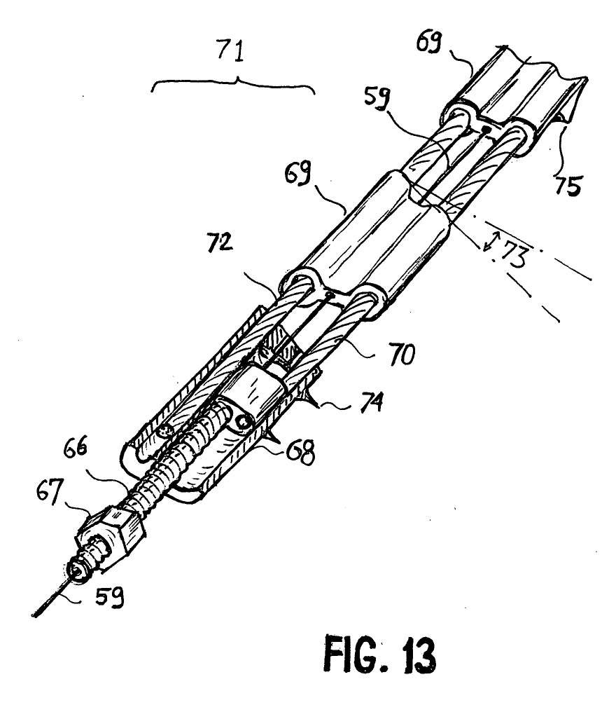

The preferred embodiment is shown in FIG. 11, FIG. 12 and FIG. 13.

The procedure is based on a chain-like device that can be inserted into the coronary sinus in its flexible state, and then made rigid and adjustable. The device is shown in FIG. 12, with a more detailed view in FIG. 13. The method of use is shown in FIG. 11.

Referring first to FIG. 12 and FIG. 13, a chain-like device 71 is made of rigid links 69 connected by two flexible cables, 70 and 72. Each one of links 69 is shaped like a trapeze. Cable 70 is connected at one end to screw 66 passing through link 68, and is also anchored to the last link at other end of chain. When nut 67 is turned cable 70 is pulled, causing the chain to move from loose and flexible shape 71B to a rigid shape 71A This is caused by the fact that in shape 71B the cables are slack and the links 69 can be flexed in all directions. When cable is tightened links 69 touch each other at the wide part of the trapezoidal shape, and start pivoting inwards around the pivot point. When edges of links 69 are in full contact, chain becomes fully rigid. The shape of the chain can be adjusted by changing the tension on cable 70, as leaving a small wedge-shaped space between links will allow a wider arc to be formed.

In order to change chain from flexible to rigid form, and to adjust the approximation of the mitral valve, a flexible tool is used. The tool comprises of a flexible outer sheath 77, flexible inner sheath 60, and guide wire 59. The guide wire is desired but nor essential. The end of the inner sheath 60 terminates in a hexagonal socket 80 which matches nut 67. The end of outer sheath 77 terminates in an oval socket 79 which matches the shape of link 68. This is needed to prevent link 68 from rotating when nut 67 is tightened. Clearly the choice of socket shapes is not important and any shape that can prevent rotation can be used. Sockets 79 and 80 can be decoupled from chain 71 simply by retracting them.

Referring now to FIG. 13, more construction details of chain 71 are shown. Cable 70 is the tensioning cable, permanently attached to screw 66 sliding inside link 68. The shape of the screw prevents is from rotating inside link 68 when nut 67 is turned. Cable 72 is an idler cable, the purpose of which is to align the links. Both cables are permanently anchored to the last link (not shown) at the chain end opposite to link 68, however cable 72 is not attached to link 68 and can slide in and out. Each link 69 has three holes: two for the cables and one for the optional guide wire 59. The cross-section of the links 69 is designed to allow blood flow in the coronary sinus above and below the links.

The ends of link 69 are not parallel to each other but form a trapezoidal shape with an angle 73. These angles (which are made different on different links) define the final shape the chain will assume. Further tightening of cable 70 after the final shape was reached only makes the cable more rigid. Link 68 and the link adjacent to it have larger angles, in order to form a sharp bend in the chain at the point it emerges from the coronary sinus. Link 68 can optionally be equipped with sharp barbs 74 in order to prevent is from sliding sideways once it reached final position. Additional barbs 75 can be added to links 69 to provide better anchoring in the coronary sinus, however due to the large encircling angle of the device in its final position it is mechanically locked in position and not likely to slide out. The advantage of not using barbs 75 inside the coronary sinus is that the device is easier to remove in case procedure needs to be reversed. To remove chain 69 the tension on cable 70 simply has to be released, causing the chain to revert to its flexible state, making it easy to pull chain out of the coronary sinus.

By the way of example, all parts of chain 71 can be made of type 316 stainless steel or of titanium. The cables are 0.8 mm diameter and the cross section of the chain is about 1.4 mm×3.5 mm. The links are made progressively smaller the farther they are from link 68, in order to better fit the coronary sinus. The screw 66 is 2 mm in diameter×20 mm long. Each link is about 10 mm long. It was found that with those dimensions the force needed to compress the mitral valve was easily achieved. Referring back to FIG. 12, the flexible sections 60 and 77 of the adjustment tool were made from bellows shaped stainless tubing having outside diameter of 4 mm and 5 mm. The rigid sections are made from regular stainless tubing of similar diameters. This allows the whole procedure to be performed via a reasonably small catheter of slightly over 5 mm inside diameter. As mentioned before, all devices described in this disclosure can be coated with special coating to make them more bio-compatible. Such coatings include, but are not limited to, drug eluting coatings.

By the way of example, a percutaneous procedure using this device is shown in FIG. 11. A catheter 78 is inserted into the right atrium 30 through the superior vena cava 22. A guide wire 59 is inserted first and pushed into the coronary sinus 20. Flexible chain 71, held by flexible tool 60 and 77, is then guided by wire 59 into the opening of the coronary sinus 15. When chain 71 reaches the desired location in the coronary sinus 20, chain is tightened by holding handle 76 and turning the inside flexible tube 60. This turns the nut pulling the steel cable (seen in FIG. 12). Note that bellows shaped tubing are very flexible for bending but can transmit a significant amount of torque. The torque needed to rotate inner tube 60 is quite low, because of the mechanical advantage of the screw. As the chain takes its desired shape, flexible tube 77 will bend and follow it. During the procedure is desired to monitor the operation of the mitral valve so to use the optimal amount of approximation. A good way of such monitoring is ultrasound Doppler velocitometer, which is a common procedure in cardiac surgery. After the correct adjustment is achieved the adjustment tool is removed by first pulling out the inner tube 60 while holding the rigid part 60 of the external tube; then pulling on the outer tube. In order to facilitate removal, a flexible tube (not shown) can be pushed into tube 77 after the removal of tube 60. This tube will push out chain 71 from the socket at the end of tube 77 without needing to pull on tube 77. This is desired as flexible tube 77 may end up at an odd angle relative to catheter 78, and it is easier to push it off end of chain 71 than to pull it off. The final shape of chain 71 is similar to the shape shown in FIG. 9, having a semi-circular portion anchored inside the coronary sinus and a more straight portion pressing against the atrial septum. Since the part inside the coronary sinus encircles close to a full semi-circle, the device is anchored in place by the virtue of its geometry.

If the device has to be adjusted (or removed) at a later date, a similar procedure to the one described above can be used. Referring now to FIG. 11 and FIG. 12, A catheter 78 is inserted into the right atrium toward the atrial septum. The larger flexible tube 77 is inserted first and guided, via fluoroscopy, ultrasound or any other means, to slide over screw 66 and then over link 68. The inner flexible tube 60 is then inserted and is guided by the outer tube 77 to mesh with nut 67. At this point re-adjustment is possible by turning inner tube 60. If device needs to be removed, nut 67 is loosened to return chain 71 to a fully flexible state. At the point inner tube 60 is removed and replaced with a similar tube having a female thread (not shown) at its end instead of socket 80. This is threaded onto screw 66. Now the chain 71 can be pulled out.

An alternative method of attachment between flexible tubes 60, 77 and chain 71 is to make link 68 of a magnetic material, such as series 400 stainless steel, and make socket 79 a strong magnet, such as by the use of rare-earth magnets. This will help in placing tool 77 back in place as the magnetic field will direct socket 79 to link 68. This also allows removal without use of a threaded tool.

FIG. 15 shows a four-chamber tomographic view through the aortic root 65 showing the location of the compression end member in relation to the interventricular septum 52 and atrioventricular septum 51. This is to more particularly point out the novelty of the current approach of percutaneous reduction of anterior-posterior diameter of a mitral valve by positioning a first end member of a compression device inside the coronary sinus while placing a second end member at the extent of the tendon of Todaro 18 in the right atrium 30.

The device of the preferred embodiment is shown in use for mitral valve approximation, however such a device is useful in other percutaneous surgical procedures, wherever there is a need to have an elongate member that can be inserted via a catheter in a flexible state and changes to a rigid adjustable state after placement in the body. Such a device can be used to support, compress, adjust and correct many internal organs. The device can be made is a large range of sizes, both in length and cross section and a large range of forms. The final shape can easily be determined by the shape of the individual links.

From the foregoing description, it should now be appreciated that a device system and methods for effecting percutaneous reduction of anterior-posterior diameter of a mitral valve has been disclosed. While the invention has been described with reference to a specific embodiment, the description is illustrative of the invention and is not to be construed as limiting the invention. Various modifications and applications may occur to those who are skilled in the art, without departing from the true spirit and scope of the invention, as described by the appended claims.

What is claimed is:

1. A method for treating mitral regurgitation using an elongate element having a first end and an opposite second end, comprising steps of:
deploying the first end in a coronary sinus;
deploying the second end in a right atrium, and
effecting an approximation of a septal annulus and a lateral annulus of the mitral valve.

2. A method as in claim 1 wherein said approximation is adjustable after elongate member is in place.

3. A method as in claim 1 wherein said elongate element has a flexible state and a more rigid state.

4. A method as in claim 1 wherein said elongate member has a flexible state and a more rigid state, and said approximation is adjustable in the more rigid state.

5. A method as in claim 1 wherein said elongate element is made of an elastic material.

6. A method as in claim 1 wherein said elongate element comprises of a plurality of rigid parts.

7. A method as in claim 1 wherein said approximation is adjusted by bending said elongate element.

8. A method as in claim 1 wherein said approximation is adjusted by changing tension on a cable.

9. A method as in claim 1 wherein said element is stepwise adjusted by using a detent action.

10. The method of claim 1 wherein said elongate member is introduced and adjusted percutaneously via a catheter.

11. A device for re-shaping body organs percutaneouly, said device having a flexible state and an adjustable more rigid state, said flexible state is used during the insertion into the body and said more rigid state is used to adjust the final shape of the device.

12. A device as in claim 11 wherein said device in made of rigid links held together be a flexible member.

13. A device as in claim 11 wherein said device is adjustable at a later date.

14. A device as in claim 1 wherein said first end and said second end can be separated for ease of insertion, to be joined and adjusted after in place.
